(19) United States
(12) Patent Application Publication
(10) Pub. No.: US 2020/0214639 A1
(43) Pub. Date: Jul. 9, 2020

(54) MEDICAL DEVICE FOR APPLYING PRESSURE TO A HUMAN JOINT SEGMENT, CORRESPONDING SYSTEM, ASSOCIATED METHOD AND USES

(71) Applicants: Universite De Poitiers, Poitiers (FR); Centre National De La Recherche Scientifique, Paris (FR); Centre Hospitalier Universitaire De Poitiers, Poitiers (FR); Universite De Tours, Tours (FR); Centre Hospitalier Regional Universitaire De Tours, Tours (FR)

(72) Inventors: Arnaud Germaneau, Mignaloux Beauvoir (FR); Tanguy Vendeuvre, Poitiers (FR); Christian Bonnard, Saint Pierre Des Corps (FR); Francois Batisse, Poitiers (FR)

(21) Appl. No.: 16/628,475

(22) PCT Filed: Jul. 4, 2018
(86) PCT No.: PCT/EP2018/068065

§ 371 Date: Jan. 3, 2020

(30) Foreign Application Priority Data
Jul. 4, 2017 (FR) 1756289

(51) Int. Cl. A61B 5/00; A61B 90/57

(52) U.S. Cl. CPC A61B 5/702; A61B 2090/061; A61B 5/4538; A61B 90/57

(57) ABSTRACT

A medical device for applying pressure to a joint segment of a human body, a corresponding system and associated uses. The device includes at least three bearing units intended to be attached to a holder. Each bearing unit has: an extendable arm; a force-measuring element attached to the extendable arm; a movement-measuring element; a bearing element which is attached to the distal portion of the extendable arm, is adapted to be in contact with a part of the human body and makes it possible to apply the force measured by the force-measuring element to the part of the human body; and an attachment element adapted to attach the bearing unit to the support.

### 1. CROSS-REFERENCE TO RELATED APPLICATIONS

This application is a Section 371 National Stage Application of International Application No. PCT/EP2018/068065, filed Jul. 4, 2018, the content of which is incorporated herein by reference in its entirety, and published as WO 2019/008020 on Jan. 10, 2019, not in English.

### 2. FIELD OF THE INVENTION

The invention relates to a medical device for applying pressure to a human joint segment. The invention also relates to a corresponding system, associated method and uses.

### 3. PRIOR ART

A joint is a junction zone between two bone ends. Joints are more or less mobile according to their constitution, their shape and the nature of the surrounding elements.

Constitutional or post-traumatic joint deformities are often a source of discomfort, pain and/or present a greater risk of injury. These deformities can also be monitored by X-ray radiography. In particular, the study of the possibility for a joint to perform movements either of abnormal amplitude, or that do not exist in a natural state—also referred to as joint laxity—is essential to better understand the joint movements of a patient. It is a very important step before any corrective action, including surgical correction.

Amongst particularly frequent joint abnormalities are sinuous deviations of the spine, also referred to as scoliosis, when the deformities occur in all three planes. Adolescent idiopathic scoliosis (AIS) can generally be corrected surgically. The purpose of surgery in this case is to obtain a balanced, stable spine, centred above the pelvis, achieving minimal vertebral fusion, while maintaining maximum adjacent mobility.

The evaluation of the reducibility of AIS curvatures is an obligation in the preoperative assessment to plan the instrumentation and arthrodesis levels. There are numerous preoperative radiographic methods for assessing the reducibility of curvatures, including supine side bending radiographs, in standing or lying position, fulcrum bending radiographs and Changai fulcrum bending radiographs, traction radiographs with or without general anaesthesia, push prone radiographs, which can be associated with traction radiographs, as well as suspension radiographs.

Supine side bending radiographs are currently considered as the benchmark preoperative radiographic method. Some surgeons, however, remain reluctant to use them because of the need for active patient participation, making the procedure inaccurate, unreliable and poorly reproducible. This method has also demonstrated its predictive effect for results with Harrington instrumentation, although no study has proven their efficiency with more modern instrumentation.

A system comprising three or four bearing units positioned on the lateral edges of a horizontal radiographic table, and making it possible to study the flexibility of a spine of a subject and/or plan the type of correction to apply by bending in three or four points of contact, was described in patent US 2011/0170671. Each bearing unit seems to comprise an extendable arm whose distal portion features a pad intended to come in contact with a part of the human body. The extendable arm has motion/positioning sensors and the pad comprises three pressure sensors. This technology advantageously aims to quantify the arm's movement as well as the pressure applied locally to the pad. However, these measurements do not appear to be able to perform a simple and quantitative analysis of the flexibility of the spine from a mechanical perspective, because this system does not give access to the reaction forces generated on each arm. Furthermore, the reproducibility of measurements over time on the same subject seems difficult to achieve. Finally, the pressure sensors are located in the X-ray field when taking an X-ray radiograph, which implies a significant overdose of X-rays in comparison with the same radiograph taken without a pressure sensor inside the pad.

Thus, none of the current preoperative radiographic methods for assessing the reducibility of AIS curvatures is fully satisfactory to practitioners.

### 4. SUMMARY OF THE INVENTION

This invention relates to a medical device for applying pressure to a joint segment of a human body, comprising at least three bearing units intended to be attached to a holder.

Each bearing unit comprises:
an extendable arm;
a force-measuring means attached to said extendable arm, adapted to measure a force applied to the extendable arm;
a movement-measuring means, adapted to measure the movement of the extendable arm;
a bearing element attached to the distal portion of the extendable arm, adapted to be in contact with a part of the human body and making it possible to apply the force measured by the force-measuring means to the part of the human body; and,
an attachment means, adapted to attach said bearing unit to the holder.

“Joint segment” means a joint or group of joints adjacent to one another on a body segment.

Thus, the at least three bearing units of the device according to the invention may be positioned on a holder and are intended to apply a force to several parts of a human body, thus making it possible to apply pressure to a joint segment. The extendable arm of each bearing unit makes it possible to apply a bearing force to a part of the human body when the bearing unit is in contact with this part of the human body at the level of the bearing element. The force-measuring means and the movement-measuring means make it possible to quantitatively measure the force applied to this part of the human body in relation to the amplitude of movement of the extendable arm. The force-measuring means attached to the extendable arm according to the invention has the advantage, compared with pressure sensors used in prior art, to make possible, reliable and reproducible the measurement of the force, by completely avoiding the contact surface of the human body with the bearing element. The device according to the invention therefore makes it possible to quantify, in a precise, reliable and reproducible manner, the movements and forces (resultants and moments) applied to a joint segment subjected to a pressure in a subject that can be in different positions, in particular lying, standing or sitting.

At least three bearing units make up the device according to the invention. Indeed, three bearing units intended to be attached to the holder make it possible to apply simple bending pressures to human joint segments. Four bearing units can be advantageously used to apply multiple bending pressures to a human joint segment. The use of four bearing units is particularly appropriate for applying complex pressures, especially on highly deformed spines, in particular spines diagnosed with scoliosis having a curvature and reverse curvature.

Each bearing unit of the device according to the invention comprises an extendable arm. The extendable arm makes it possible to exert a force on a part of the human body when the bearing unit is in contact with this part of the human body at the level of the bearing element. For example, for a joint segment being the spine, the extendable arm makes it possible to exert a force on a part of the trunk, in particular on the upper, middle or lower part of the trunk, in a frontal plane or in a sagittal plane.

The extendable arm comprises any means for exerting a thrust. Advantageously, the extendable arm can generate a bearing force of between 0 and 1000 Newtons.

Preferably, the extendable arm comprises a rod intended to be guided longitudinally in translation in a rectilinear direction. The extendable arm can then extend in a rectilinear direction. The fact that the force is exerted along the axis of the rod is particularly advantageous insofar as the force exerted on the part of the human body, after precisely positioning the direction of the rod, is then purely a compressive force of the rod. The force vector exerted on the part of the human body in contact with the bearing element is then perfectly characterised since its direction, position and norm are perfectly established. The fact of operating the extendable arm in pure compression makes it possible to better control, at least to control more easily, the good reproducibility of the movement and force measurements for a given subject, as well as the analysis of the mechanical properties, in particular flexibility, of their spine.

According to a first embodiment, the rod is guided by a connection such as a slide or sliding pivot connection. In this embodiment, the extendable arm can also be a manual push rod guided by a slide type connection or a sliding pivot type connection, a pneumatic cylinder, an electric cylinder or a hydraulic cylinder.

According to a second embodiment, the rod is guided by a helical type connection. In this embodiment, the extendable arm can in particular be a screw-nut system or a ball screw system. The screw-nut system can be actuated manually or can be motorised.

Each bearing unit of the device according to the invention comprises a force-measuring means. For a given bearing unit, this force-measuring means is attached to the bearing arm and makes it possible to measure the force exerted on the extendable arm. As mentioned above, the force-measuring means according to the invention has the advantage over the pressure sensors used in the prior art, of making the measurement of reaction force possible in a reliable and reproducible manner, by completely avoiding the contact surface of the human body with the bearing element.

The resolution of the force-measuring means is preferably at least of the order of the Newton.

The force-measuring means may in particular be selected from: strain gauge force sensors, piezoelectric force sensors, “S type” force sensors, torquemetres, flange mount force sensors and hollow cylindrical force sensors.

Advantageously, the force measuring means is a hollow cylindrical compression force sensor. The hollow cylindrical force sensor is attached to a part of the extendable arm intended to be stationary, as well as to the rest of the bearing unit structure, also intended to be stationary. The use of a hollow cylindrical compression force sensor has in particular the advantage of making it possible to move the force-measuring means away from the zone in which the pressure is applied and to reduce the size of the bearing unit, while ensuring the measurement of the pure compressive force applied inside the rod. The hollow cylindrical compression force sensor may in particular be positioned at a suitable distance from the zone where the pressure is applied so as not to interfere with the X-ray radiography instrumentation. Indeed, the presence of the force-measuring means, chiefly made of metallic material, in the X-ray radiography field, would substantially change the calibration of the X-ray radiography instrumentation (increase in X-ray beam power during radiography) and would imply unnecessary overexposure of the patient to X-rays.

Each bearing unit of the device according to the invention comprises a movement-measuring means. The movement-measuring means is adapted to measure the movement of the extendable arm. The movement-measuring means may in particular be selected from: a graduated scale, a graduated dial, a laser displacement sensor, a probe, a comparator, a non-contact sensor. Preferably, the resolution of the movement-measuring means is of the order of one millimetre.

Advantageously, the movement-measuring means is positioned at a suitable distance from the zone where the pressure is applied so as not to interfere with the X-ray radiography instrumentation. According to a special embodiment of the invention, the extendable arm is a screw-nut system, the screw being intended to extend in a rectilinear direction, the nut remaining stationary relative to the holder. In this embodiment, the movement-measuring means is advantageously a screw revolution counter, the movement obtained for a complete revolution of the screw corresponding to the screw pitch.

Each bearing unit of the device according to the invention also comprises a bearing element attached to the distal portion of the extendable arm. The bearing element is adapted to and intended to come in contact with a part of the human body. The bearing element may advantageously be made of a flexible polymer. In addition, the bearing element may in particular have a shape adapted to the body segment of the human body on which it is intended to constitute a pressure. An adapted shape is in particular a shape that allows suitable distribution of the forces over a suitably large surface of a human body segment. The bearing element can have a wide variety of shapes such as a flat shape, a cylindrical shape, a spherical shape, a convex shape or a concave shape.

Finally, each bearing unit of the device according to the invention comprises an attachment means adapted to attach the bearing unit to a holder. The nature of the attachment means depends on the nature of the holder. In a specific embodiment, the attachment means is removable and may in particular be selected from: a vice, a staple, a flange, a collar, a clamp, a seal clamp and a spring clip. Advantageously, the attachment means comprises a spring clip. This also has the advantage that the bearing units of the device according to the invention can be easily and quickly attached to, or removed from, the holder.

According to an advantageous embodiment of the invention, each bearing unit also comprises a pivot connection or sliding pivot connection along an axis. The pivot connection or sliding pivot connection makes it possible to adjust the position of the extendable arm and/or the bearing element angularly or vertically with respect to said axis. The locking element reversibly locks the angular and vertical position of the extendable arm and/or bearing element. Advantageously, each bearing unit also comprises an angular measuring means of the position of the extendable arm and/or bearing element with respect to said axis. This advantageously makes it possible to precisely measure the direction of the bearing force. The angular measuring means may in particular be selected from: a graduated dial or a digital goniometer. Preferably, the resolution of the angular measuring means is of the order of one degree.

According to a preferred characteristic of the invention, elements of the device intended to be in the X-ray field of an X-ray radiography are made of radiolucent materials. Radiolucent materials do not or hardly appear on X-ray radiographic images. Radiolucent materials are in particular polymers selected amongst polyamides, polytetrafluoroethylenes (PTFE), poly(methyl methacrylates) (PMMA), polyurethanes (PU), polycarbonates (PC), polyvinyl chlorides (PVC), polyethylenes (PE), polymer foams and elastomer polymers. Elements of the device intended to be in the X-ray field of an X-ray radiograph are in particular the extendable arm, in particular its distal end, and the bearing element of each bearing unit. This makes it possible in particular to take an X-ray radiograph with bearing units without involving a significant overdose of X-rays in comparison with the same radiograph without the bearing units, while making it possible to achieve a good quality radiographic image.

The invention also relates to a system for applying pressure to a joint segment of a human body. The system comprises a medical device as described above and a holder. The bearing units are attached to the holder in a removable or non removable manner. At least one of the bearing units is intended to constitute a pressure against a part of the human body. At least another of the bearing units is intended to constitute a counter-pressure against another part of the human body.

“Counter-pressure” is meant as a force having a direction noticeably opposite to a “pressure”.

The holder is adapted according to the nature of the human joint segment and the patient.

The holder may in particular be an X-ray table, an examination table, an operating table, in the horizontal, reclined or vertical position or a frame, a wheelchair, in the case of a joint segment being the spine.

The holder may in particular be a wheelchair, an ergometer, a tablet adapted or not adapted to radiology, in the case of a joint segment selected amongst joints of the wrist, elbow, shoulder, hips, knee and ankles.

According to a special embodiment of the invention, the holder is a noticeably flat holder. “Noticeably flat holder” means a holder whose surface is apparently flat, despite any potential roughness and any other defects said surface may present. According to this embodiment of the invention, the system for applying pressure to a joint segment of the human body comprises: a medical device for applying pressure to a human joint segment comprising at least three bearing units and a noticeably flat holder. The bearing units are attached to the lateral edges of the noticeably flat holder. Each bearing unit preferably comprises:
an extendable arm adapted to extend in a rectilinear direction in a noticeably parallel plane to that of the noticeably flat holder;
a force-measuring means attached to said extendable arm, adapted to measure the force applied to the extendable arm in said rectilinear direction;
a movement-measuring means, adapted to measure the rectilinear movement of the extendable arm;
a bearing element attached to the distal portion of said extendable arm, adapted to be in contact with a part of a human body and making it possible to apply the force measured by the measuring means to the part of the human body; and,
an attachment means, whereby the bearing unit can be attached to the lateral edges of the holder;

According to this embodiment, each bearing unit of the device also advantageously comprises: a pivot connection or sliding pivot connection along a noticeably orthogonal axis with respect to the plane of the holder, making it possible to adjust the position of the extendable arm and/or the bearing element angularly and/or vertically along said axis, a locking element also making it possible to reversibly lock the angular and/or vertical position of the extendable arm and/or bearing element.

According to this embodiment, the attachment means of each bearing unit is preferably removable, so that each bearing unit can be adjusted in position on the lateral edges of the holder according to the morphology of the human body.

The invention also relates to a method for applying pressure to a joint segment of a human body. The method comprises a step for positioning the bearing units of a system according to this invention, as described above. The step for positioning the bearing units is performed so as to place the bearing elements of said bearing units in contact with parts of the human body. The method also comprises a pressure application step of at least one bearing unit to at least one part of the human body. This pressure application step is controlled by measuring the movement of the extendable arm and by measuring the force exerted on the extendable arm of each of said bearing units. The method according to the invention therefore makes it possible to quantify the pressure applied to a joint segment in a precise, reliable and reproducible manner.

Advantageously, the pressure application step is also controlled by measuring the angular position of the extendable arm.

The invention also relates to the use of a system as described above to study the ligament laxity, or flexibility, of a human joint segment. The human joint segment can in particular be selected from the joints of the wrist, elbow, shoulder, spine, hips, knee and ankle. Due to the good mastery of the accuracy and reproducibility of the movement and force measurements of the extendable arm of the bearing units, as well as the good control of the direction of the forces exerted by the bearing units, an accurate and reproducible analysis of the flexibility and mechanical behaviour of the joint segment is possible.

Specifically, and as described in detail in the non restrictive example of embodiment of the invention, the invention also relates to the use of a system as described above to predict in a preoperative manner the expected surgical correction for a human spine with a deformity.

### 5. LIST OF FIGURES

The invention, as well as its various advantages, will be more readily understood with the following description of a non restrictive embodiment of this invention, given in reference to the drawings, wherein:

FIG. 1A and FIG. 1B show a bearing unit of a device, according to this invention, attached to a holder and intended in particular to constitute a pressure against a part of the trunk; FIG. 1A is a perspective view of the bearing unit, whereas FIG. 1B is a cross-sectional view of the same bearing unit.

FIG. 2 is a top view of a system comprising i) a device comprising three bearing units as shown in FIG. 1A and ii) an X-ray table.

FIGS. 3A and 3B show a system comprising i) a device comprising four bearing units as shown in FIG. 1A and ii) a holder being an X-ray table (top view, FIG. 3A) or a frame (front view, FIG. 3B).

FIG. 4 shows an appropriate positioning of four bearing units on a holder in the case of a spine with two curvatures.

FIG. 5 shows an appropriate positioning of four bearing units on a holder in the case of a spine with one curvature.

FIGS. 6A, 6B and 6C show X-ray radiographic images of the trunk of a patient in the frontal plane, without pressure (FIG. 6A), with right side bending (FIG. 6B, “Bending R”) and with left side bending (FIG. 6C, “Bending L”).

FIGS. 7A, 7B and 7C show X-ray radiographic images of the trunk of a patient in the sagittal plane, without pressure (FIG. 7A), with forward bending (FIG. 7B) and with backward extension (FIG. 7C).

### 6. DETAILED DESCRIPTION OF AN EMBODIMENT

### 6.1 Example of a Bearing Unit Adapted for Applying Pressure to the Spine

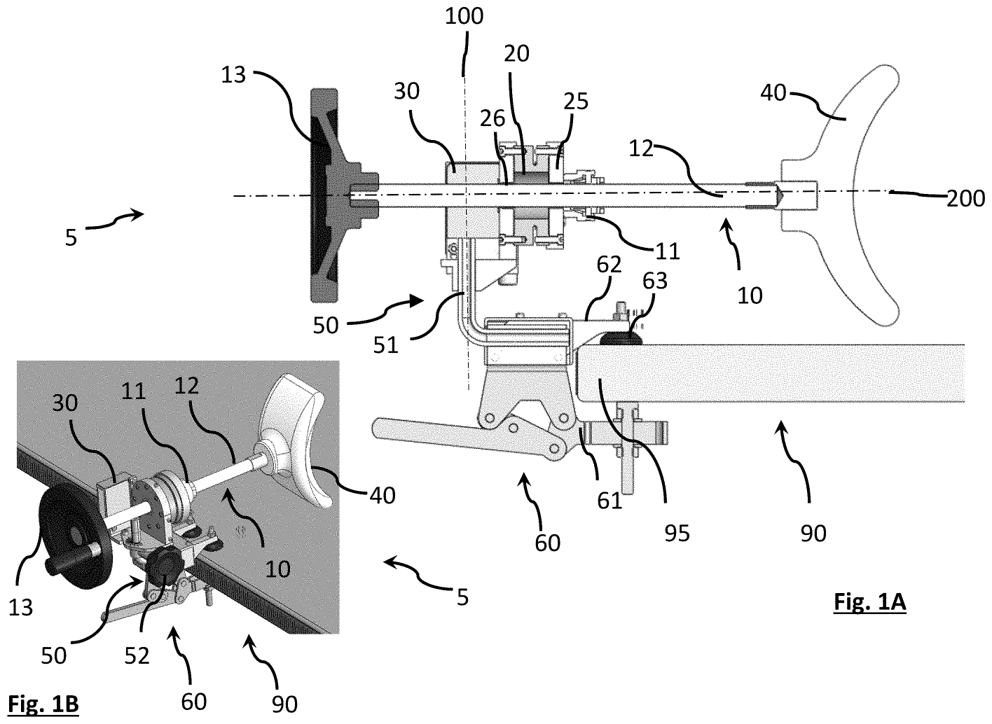

In reference to FIG. 1A and FIG. 1B, a bearing unit 5 can be attached in a removable manner to the lateral edges 95 of an X-ray table 90. Each bearing unit 5 comprises: an extendable arm 10, a hollow cylindrical compression force sensor 20, a laser displacement sensor 30, a bearing element 40 and an attachment means 60.

Extendable arm 10 comprises a steel bearing 25, a nut 11, a screw 12 having an axis 200 and a crank 13. Nut 11 is normally secured to bearing 25. Crank 13 is attached to the proximal end of screw 12, and makes it possible to turn screw 12 inside nut 11: screw 12 can therefore be moved in a rectilinear direction, the nut remaining stationary with respect to the rest of bearing unit 5. Screw 12 used is of the M16 type and has a screw pitch of 2 mm. The screw 12-nut 11 system of extendable arm 10 has many advantages: the practitioner can easily handle it and can get quite a precise idea of the movement by counting the number of revolutions of crank 13, without even having to refer to laser displacement sensor 30. Moreover, for a given position of screw 12, wherein a longitudinal force is applied to screw 12, the position of the screw remains unchanged, regardless of the force applied: this ensures in particular good accuracy and reliability of the movement applied by the practitioner to extendable arm 10, as well as good repeatability of the force exerted by extendable arm 10 on the same patient for a given part of the body.

Nut 11 can temporarily be uncoupled from bearing 25 so as to allow the practitioner to roughly position screw 12 in a required position by simple thrust, without having to apply too many revolutions to crank 13. Bearing 25 also has a Teflon coating 26 at the points that can come in contact with screw 12: this guarantees good sliding pivot connection between screw 12 and bearing 25 when nut 11 is temporarily uncoupled from bearing 25.

Hollow cylindrical compression force sensor 20 is secured to bearing 25 and makes it possible to measure the force exerted on screw 12 along axis 200. It has a resolution of at least of the order of one Newton.

Laser displacement sensor 30 is also secured to bearing 25 and makes it possible to measure the amplitude of the rectilinear movement of screw 12 along axis 200.

Bearing member 40 is attached to the distal portion of screw 12. It has a concave shape adapted to be in contact with parts of the trunk of a human body, in particular the upper, middle or lower parts of the trunk, in the frontal plane or in the sagittal plane. The concave shape allows for good distribution of the pressure applied by extendable arm 10 to any part of the trunk. Naturally, for the application of pressure to other joint segments, bearing element 40 can have a different shape and a different size according to the part of the body to which the application of pressure is considered.

Attachment means 60 is a removable attachment means. It comprises: a horizontal spring clip 61, attached to a lateral profile 62 having a base 63. Attachment means 60 advantageously makes it possible to easily and quickly attach or remove bearing unit 5 to or from X-ray table 90. A graduated scale (not shown), may also be positioned along lateral edges 95 of X-ray table 90 so as to determine the transversal position of bearing unit 5 on lateral edges 95 of X-ray table 90. An alternative (not shown) attachment means 60 could be a sliding rail attached to lateral edges 95 of X-ray table 90, making it possible to adjust the transversal position of bearing unit 5 and, a locking means, for example a screw, making it possible to lock the bearing unit in a given transversal position.

A sliding pivot connection 50, between attachment means 60 and bearing 25 makes it possible to adjust the height and angular position of extendable arm 10 with respect to a baseline value 100 to X-ray 90. Sliding pivot connection 50 is formed by a long centring on a bent tube 51. Locking of extendable arm 10 at a given height and in a given angular position is achieved by tightening a screw on the bent tube through a six-lobe wheel 52. The height and angular position of extendable arm 10 can be measured by graduated dials (not shown).

Since the X-ray radiography acquisition system calibrates its radiation power automatically according to the density of the materials to be crossed, bearing 25, nut 11, hollow cylindrical compression force sensor 20 and laser displacement sensor 30 are arranged so that when bearing unit 5 is attached to lateral edges 95 of an X-ray table 90, they are also positioned towards lateral edges 95 of X-ray table 90, preferably outwards. Bearing 25, nut 11, hollow cylindrical compression force sensor 20 and laser displacement sensor 30 are then out of the X-ray field when taking an X-ray radiograph. Moreover, the elements being intended to be in the X-ray field when taking an X-ray radiograph, in particular screw 12 and bearing element 40, are made of radiolucent materials. Screw 12 is made of polyamide. Bearing element 40 is made of polymethylmethacrylate. Thus, the elements that would significantly absorb the X-rays are moved outside the X-ray field, elements that would absorb few of the X-rays being used in the X-ray field. This allows in particular for an X-ray radiograph with bearing units 5 attached to X-ray table 90 to be taken without involving a significant overdose of X-ray compared to the same radiograph without bearing units 5, while providing a good quality radiographic image. Thus, the device comprising three or four bearing units 5 makes it possible to apply pressure to a spine in a given shape and to have personalised quantitative data on the forces and movements measured during X-ray radiography. Bearing forces 5 are made with simple elements, adaptable to all types of holder, in particular X-ray tables. It can also be considered that a patient in a wheelchair can undergo sitting stress radiography in their wheelchair.

### 6.2 Example of a System Adapted for Applying Pressure to the Spine.

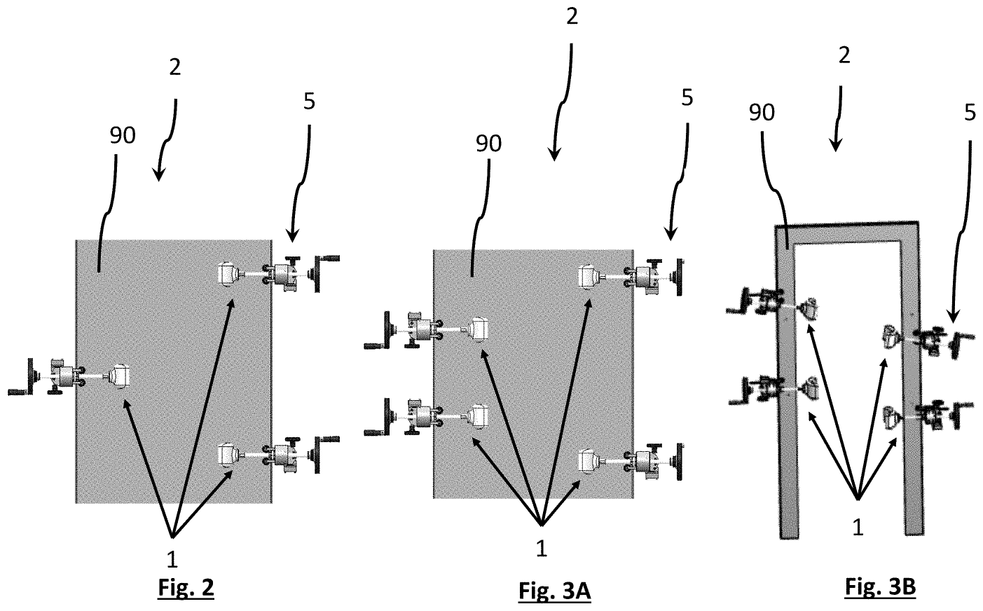

In reference to FIGS. 2 and 3A, a system 2 comprises: a device 1 comprising three (see FIG. 2) or four (see FIG. 3A) bearing units, as described in section 6.1, attached to an X-ray table 90.

In the case of three bearing units 5 (see FIG. 2), the central bearing unit 5 is arranged to constitute a pressure and the two end bearing units 5 are arranged to constitute a counter-pressure (or vice versa).

The use of four bearing units compared to three bearing units has several advantages. The pressure applied is more homogeneous between the bearings: there are fewer pressure variations generated in the joint segment, and thus less loading singularity for the patient, facilitating the correction and application of pressure to the joint segment (greater loading acceptability and tolerance for the patient); there is also greater interchangeability of the shape and size of the four bearing points without affecting the load and measurement quality. The positions of the four bearing points are modifiable independently and adjustable according to the patient's morphology and the required deformation of the joint segment.

In particular, in the case of pressure applied to the spine, the four bearing units 5 can be arranged differently according to the type of deformation.

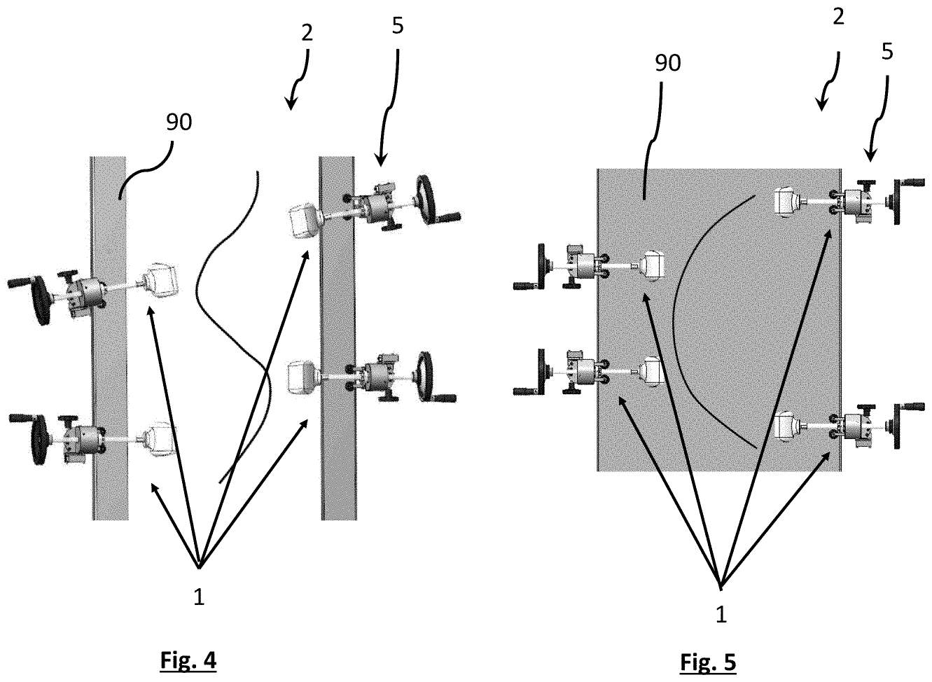

In reference to FIG. 4, in the case of a spinal deformity with two curvatures (one curvature and one counter-curvature), the four bearing units 5 can be positioned as follows so as to achieve a straightening of the spine:
a bearing unit 5 on each of the two vertices of curvature;
a bearing unit 5 at the proximal end of the overall deformation; and,
a bearing unit 5 at the distal end of the overall deformation.

One advantage of using four bearing units over three bearing units is that, in the case of four bearing units, pressure can be applied to the curvature and counter-curvature simultaneously (only one radiographic image), whereas in the case of three bearing units, pressure must be applied to each curvature separately (two radiographic images). Furthermore, simultaneous pressure application to the curvature and counter-curvature makes it possible to take into account the effects of the correction of the curvature over the counter-curvature, and vice versa.

In reference to FIG. 5, in the case of a spinal deformity with one curvature, the four bearing units 5 can be positioned as follows so as to achieve a straightening of the spine:
two bearing units 5 placed on either sides of the vertex of the curvature;
a bearing unit 5 at the proximal end of the overall deformation; and,
a bearing unit 5 at the distal end of the overall deformation.

The configuration shown in FIG. 5 can be used for pressure application to most joints other than the spine.

The bearing units 5 are positioned according to the nature of the joint segment, the required deformations of the joint segment and the patient's physiology on suitable parts of the patient's body.

As shown in FIGS. 3A and 3B, possible holders to which the four bearing units can be attached are in particular an X-ray table (see FIG. 3A) or a frame (see FIG. 3B). The nature of the holder preferably used depends on the uses of the medical device. For example, as detailed in section 6.3, it may be advantageous to use a horizontal X-ray table on which the patient can lie in the case of use for preoperative correction of the shape of the spine, since the patient must be in a lying position during the corrective procedure. Conversely, it may be advantageous to use a vertical frame, the patient must then remain upright, in the case of use for the creation of a corset aimed to correct the shape of the spine, the corset being intended to be worn in particular in the sitting or standing positions.

### 6.3 Use of the System for Applying Pressure to a Spine in a Given Position

The system shown in FIG. 2 is used for applying pressure in a given position to the spine of a person with a spinal deformity, while quantitatively measuring the direction and value of the forces applied to each one of the extendable arms of the bearing units, as well as the movement value for each one of the extendable arms of the bearing units.

A general method that can be used for various uses of the system covered in section 6.2 comprises steps for:

i) positioning a patient in the frontal or sagittal plane;
ii) taking a first radiograph of the spine without applying pressure;
iii) issuing a diagnosis regarding the deformation of the spine from the first radiograph;
iii) positioning the bearing units in a pressure or counter-pressure against parts of the patient's body according to the diagnosis issued after the first radiograph and according to the patient's morphology;
iv) applying pressure by at least one bearing unit, in a controlled manner according to the measurement of quantitative positioning values (rectilinear positioning and movement of the extendable arm, and angular position) and of a force applied to the bearing units: the spine is then referred to as being “under pressure”; and,
v) taking a second radiograph of the spine with pressure applied.
Steps iii), iv) and v) can possibly be repeated iteratively from the diagnosis issued from the last radiographic image of a spine “under pressure”.

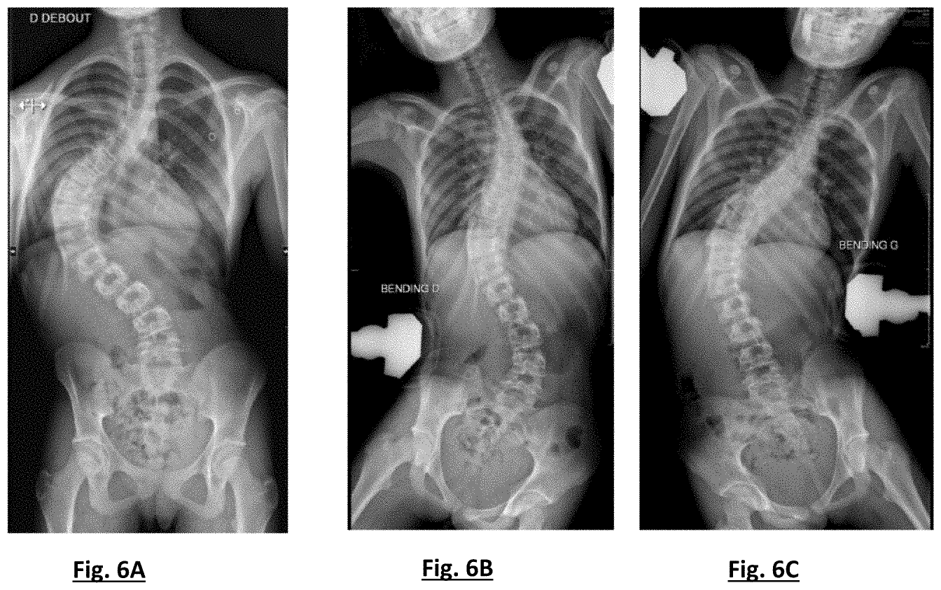

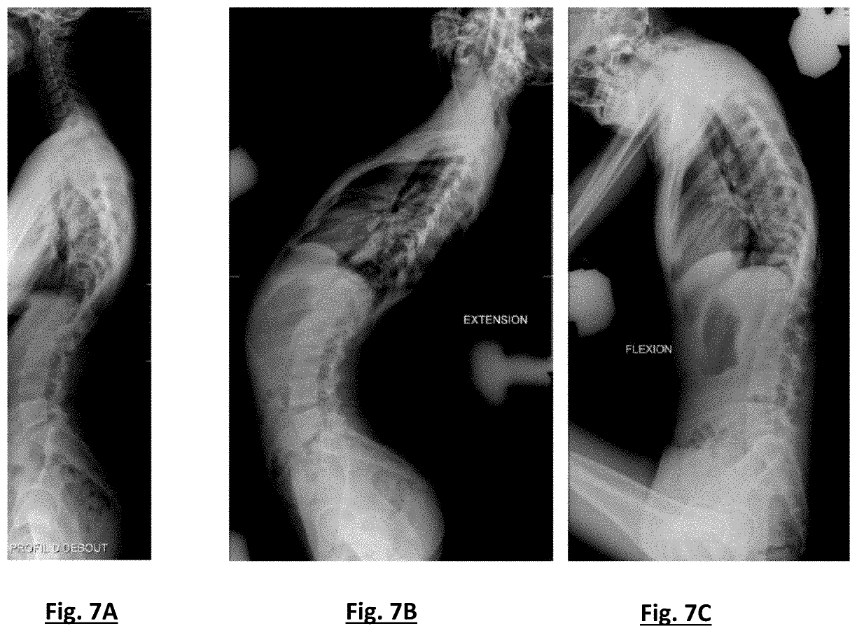

The method thus combines the use of a system according to the invention with an X-ray radiography apparatus. The X-ray radiography apparatus makes it possible to monitor the changes in the shape of the spine without pressure application or under various types of pressure (including the state perfectly defined by the measurement of quantitative values for the positioning and force applied to the bearing units). The conventional X-ray radiography apparatus implies the taking of images according to a single plane, in particular either the sagittal plane, or the frontal plane. Examples of images are shown in FIGS. 6A, 6B, 6C, 7A, 7B & 7C. FIG. 6A shows a radiographic image without pressure of a spine in the frontal plane. FIGS. 6B and 6C show radiographic images of the spine with pressure application, in respectively right and left side bending. Similarly, FIG. 7A shows a radiographic image of a spine without pressure application in the sagittal plane. FIGS. 7B and 7C show radiographic images of the spine under pressure application, in respectively forward bending and backward extension.

A more elaborate apparatus than the conventional X-ray radiography apparatus can also be used, such as an EOS imaging apparatus, making it possible to scan the frontal plane and sagittal plane and emitting a lower X-ray dose.

All the quantitative positioning and force measurements recorded by the various sensors comprised in the bearing units can be acquired via computer acquisition cards to facilitate data processing and analysis. It is therefore possible with the same computer tool to correlate the movements and forces applied to each bearing unit with the spinal deformity between the first radiographic image (spine without pressure application) and the n^{th}, in particular second, radiographic image (spine under pressure application). Thus, it is possible to correlate the biomechanical analysis of the flexibility and correction of the spinal deformity and the clinical analysis.

In general, the method described above may be used to prevent pressure ulcers related to pressure in a joint segment compression system. Indeed, pressure ulcers appear following excessive pressure applied to tissue, in particular the skin. The bearing units of the medical device in particular make it possible to identify the parts of the body where the most pressure is applied when wearing the compression system: the compression system can then be adapted to reduce excessive pressure applied locally.

In particular, the method detailed above can make it possible to consider the optimisation of the bearings for the manufacture of corsets. For such a use, radiographs are advantageously performed in the standing position so as to be as close as possible to the conditions of use of the corset. The bearing units of the medical device make it possible in particular to determine the maximum correction that can be considered, as well as the parts of the body where the most pressure will be applied when wearing the corset, in order to design a corset that induces physiologically acceptable conditions for the patient and minimising the risk of pressure ulcers.

The method detailed above may also be used to preoperatively predict the correction that can be expected by spinal surgery. Use of surgery is often necessary when significant spinal deformities (curvatures) have been diagnosed. This method is intended to help the surgeon by offering the possibility of straightening the spine temporarily, to take radiographic images in one or several corrective situations to predict the geometry that the scoliotic spine will have after surgery. This method therefore makes it possible to reduce the operative/post-operative risks induced by significant corrections. It also makes it possible to help the surgeon in the selection of the stabilisation equipment and in the positioning of the implants on each vertebra. It enables a help in the decision of the surgical approach: anterior, posterior, lateral or combined. It also makes it possible to determine the most appropriate moment for surgery.

This method is a major breakthrough in the prediction of preoperative corrections compared to the methods of the prior art. Firstly, to the inventors' knowledge, there is no such device/system allowing the performance of a spinal deformation in situ in an X-ray radiograhy apparatus. Secondly, all the predictions can be made in a physiological position, i.e. in a position analogue to the operative position. Thirdly, as the patient does not actively participate in this method, corrective predictions are more reliable, accurate and reproducible than those obtained in the methods of the prior art.

Thus, the method can be applied to a given patient, diagnosed with spinal deformity or scoliosis (first radiograph, without pressure application). One or several deformations are applied to the spine of the patient, remained alert, using the device according to the invention to seek a satisfactory connection (second or n^{th} radiograph, under pressure): the device makes it possible to obtain quantified data of this correction, and guide and optimise:
the choice of implantable medical devices;
the choice of materials to be used for rods to optimise the stiffness and resistance in relation to the considered correction: alloys, titanium, chromium-cobalt, etc.;
the number of positioning in relation to the number of stages to correct;
the sizes of the implants adapted in relation to the stages to correct; and,
implant materials.

The patient is asleep during surgery and is placed in the same corrective conditions with a similar situation for the correction (positions of the extendable arms, movement of the extendable arms, angles, applied forces). The same correction as in an alert situation is thus applied, significantly minimising the risks of overcorrection and thus, the neurological deficit during surgery.

### 6.4. Generalisation

Although the examples in sections 6.1, 6.2 and 6.3 focus almost exclusively on the spine, a generalisation of the use of the device to other joint segments is accepted, by dimensional/functional adaptation of the bearing units in relation to the joint segment of interest and by using a suitable holder to attach the device.

For example, the device may be used for the knees and makes it possible to:
identify the level of pressure of a prosthesis;
select the type of prosthesis that can be used: constrained, semi-constrained or hinged prosthesis;
quantify ligament instabilities by ensuring the contact of each of the points, irrespective of the patient's morphology and level of cooperation and participation; and
eliminate muscular stiffening during the examination.

### 7. CONCLUSION

An exemplary embodiment of this invention overcomes at least some disadvantages of current methods, whether the joint segment of the spine, as detailed above, or other joint segments, such as the joints of the wrist, elbow, knee, hip, shoulder or ankle.

An exemplary embodiment provides a medical device for applying pressure to a human joint segment in a quantified manner and within the patient's tolerance. More specifically, an exemplary embodiment provides a medical device making it possible to accurately, reliably and reproducibly apply pressure to a human joint segment.

An exemplary embodiment provides a medical device that can be used for X-ray radiography, in particular that allows taking radiography without excessive irradiation for the patient.

An exemplary embodiment provides a simple, relatively inexpensive medical device, easily adaptable with currently available medical equipment.

An exemplary embodiment offers a system comprising the medical device.

An exemplary embodiment offers a method for applying pressure to a human joint segment.

An exemplary embodiment offers specific uses for the medical device/system.

Although the present disclosure has been described with reference to one or more examples, workers skilled in the art will recognize that changes may be made in form and detail without departing from the scope of the disclosure and/or the appended claims.

1. A medical device for applying pressure to a joint segment of a human body comprising:
at least three bearing units configured to be attached to a holder, each bearing unit comprising:
an extendable arm;
a movement-measuring element, adapted to measure movement of said extendable arm;
an attachment, adapted to attach said bearing unit to said holder;
a force-measuring element attached to said extendable arm, adapted to measure a force exerted on said extendable arm; and
a bearing element attached to a distal portion of said extendable arm, adapted to be in contact with a part of the human body and making it possible to apply said force measured by said force-measuring element to said part of the human body.

2. The medical device according to claim 1, wherein the medical device comprises four bearing units.

3. The medical device according to claim 1, wherein said extendable arm comprises a rod that is guided longitudinally in translation in a rectilinear direction.

4. The medical device according to claim 3, wherein said force-measuring element comprises a hollow cylindrical compression force sensor.

5. The medical device according to claim 1, wherein each bearing unit comprises:
a pivot connection or sliding pivot connection along an axis, allowing a position of said extendable arm and/or said bearing element to be adjusted angularly and/or vertically with respect to said axis, and
a locking element allowing the angular and/or vertical position of said extendable arm and/or said bearing element to be locked reversibly.

6. The medical device according to claim 5, wherein the medical device comprises an angular measuring element, which measures the position of said extendable arm and/or said bearing element with respect to said axis.

7. The medical device according to claim 1, wherein the distal end of the extendable arm and the bearing element are made of radiolucent materials.

8. A system for applying pressure to a joint segment of a human body, the system comprising:
a holder;
a medical device, which comprises at least three bearing units attached to the holder, each bearing unit comprising:
an extendable arm;
a movement-measuring element, adapted to measure movement of said extendable arm;
an attachment, which attaches said bearing unit to said holder;
a force-measuring element attached to said extendable arm, adapted to measure a force exerted on said extendable arm; and
a bearing element attached to a distal portion of said extendable arm, adapted to be in contact with a part of the human body and making it possible to apply said force measured by said force-measuring element to said part of the human body,
wherein at least one of said bearing units is arranged to apply a pressure against the part of said human body, and at least one other of said bearing units is arranged to apply a counter-pressure against another part of said human body.

9. A method for applying pressure to a joint segment of a human body, comprising:
positioning a human body on a holder on which a medical device is attached, comprising at least three bearing units, each bearing unit comprising:
an extendable arm;
a movement-measuring element, adapted to measure movement of said extendable arm;
an attachment, which attaches said bearing unit to said holder;
a force-measuring element attached to said extendable arm, adapted to measure a force exerted on said extendable arm; and
a bearing element attached to a distal portion of said extendable arm, adapted to be in contact with a part of the human body and making it possible to apply said force measured by said force-measuring element to said part of the human body,
wherein at least one of said bearing units is arranged to apply a pressure against the part of said human body, and at least one other of said bearing units is arranged to apply a counter-pressure against another part of said human body;
positioning the bearing units so as to bring the bearing elements of said bearing units in contact with parts of said human body; and
applying the pressure of at least one of said bearing units to at least one of said parts of the human body, said applying the pressure being controlled by measuring the movement of said extendable arm and by measuring the force exerted on said extendable arm of each of said bearing units.

10. The method of claim 9, further comprising studying ligament laxity of a joint segment of a human body based on the act of applying the pressure, said human joint segment being selected from the joints of the wrist, elbow, shoulder, spine, hips, knee and ankle.

11. The method of claim 9, further comprising pre-operatively predicting a correction that can be expected by surgery for a human spine with deformity based on the act of applying the pressure.
